(19) United States
(12) Patent Application Publication
(10) Pub. No.: US 2026/0216539 A1
(43) Pub. Date: Jul. 30, 2026

(54) SYSTEMS AND METHODS FOR CONTROLLING TRANSDUCER MODULES FOR GENERATING FOCUSED ULTRASOUND

(71) Applicant: SUNNYBROOK RESEARCH INSTITUTE, TORONTO (CA)

(72) Inventor: KULLERVO HYNYNEN, TORONTO (CA)

(21) Appl. No.: 19/147,479

(22) PCT Filed: Dec. 11, 2023
(86) PCT No.: PCT/CA2023/051637

§ 371 Date: Jul. 11, 2025

Related U.S. Application Data
(60) Provisional application No. 63/438,922, filed on Jan. 13, 2023.

(51) Int. Cl. A61N 7/00

(52) U.S. Cl. CPC A61N 7/00; A61N 2007/0078; A61N 2007/0095

(57) ABSTRACT

A phased array ultrasound system includes one or more ultrasound array modules having respective array elements, each array element connected to a respective switch. Each array module receives a set of driving signals, each driving signal having a respective phase value, and the set of driving signals are provided to each switch. The switches are controlled such that the driving signal provided to a given array element has a phase value that is closest to an ideal phase value for focusing ultrasound energy at a selected focus location. At least two of the array modules each receive a unique module-specific set of driving signals having a respective unique set of phases. A given unique set of phases values may be determined based on a set of ideal phases for focusing ultrasound energy at one or more locations, such as a selected focal location or a selected focal region.

### CROSS-REFERENCE TO RELATED APPLICATION

This application claims priority to U.S. Provisional Patent Application No. 63/438,922, titled “SYSTEMS AND METHODS FOR CONTROLLING TRANSDUCER MODULES FOR GENERATING FOCUSED ULTRASOUND” and filed on Jan. 13, 2023, the entire contents of which is incorporated herein by reference.

### BACKGROUND

The present disclosure relates to ultrasound-based therapy and imaging. More particularly, the present disclosure relates to ultrasound systems and methods.

The use of ultrasound for diagnostic and therapeutic applications has been studied extensively over the last several decades. In addition to the routine use in diagnosing disease, ultrasound beams that are focused and have been found to be effective for example for tumor ablation, thrombolysis, drug delivery, gene therapy, blood vessel occlusion, local enhancement of the permeability of the blood brain barrier, and neuromodulation. Focused ultrasound as a course of treatment affords the same benefits as traditional interventions in that it provides an effective method for neutralizing the effects of pathological tissue but with minimal side effects and hugely reduced recovery time.

During the last 25 years, phased-array transducers have become prominent as a means of creating the ultrasonic fields. These devices provide electronic beam steering thus eliminating the need for moving parts as well as enabling compensation for distortions induced by the intervening tissue. In order to develop systems that exhibit full control of the focus location, the number of elements will need to be large. For full electronic focal steering the maximum center-to-center element spacing is λ/2, where λ is the wavelength. For therapeutic frequencies, which range from 0.2 to 10 MHz, this condition would require inter-element spacing about 3 mm to 0.075 mm. In order to generate a therapeutically significant amount of power and adequate focusing gain, there would have to be a large number of independently driven transducer elements.

This creates problems related to the cost and size of the cable assembly and driving electronics that provide the RF-signals.

### SUMMARY

A phased array ultrasound system includes one or more ultrasound array modules having respective array elements, each array element connected to a respective switch. Each array module receives a set of driving signals, each driving signal having a respective phase value, and the set of driving signals are provided to each switch. The switches are controlled such that the driving signal provided to a given array element has a phase value that is closest to an ideal phase value for focusing ultrasound energy at a selected focus location. At least two of the array modules each receive a unique module-specific set of driving signals having a respective unique set of phases. A given unique set of phases values may be determined based on a set of ideal phases for focusing ultrasound energy at one or more locations, such as a selected focal location or a selected focal region.

Accordingly, in a first aspect, there is provided a phased array ultrasound system comprising:
a support;
a plurality of ultrasound modules mechanically supported by the support, each ultrasound module comprising a respective array of ultrasound elements, wherein each ultrasound element is in electrical communication with an output of a respective switch uniquely associated with the ultrasound element; and
control and driving electronics configured to generate and deliver, to each ultrasound module, a respective set of driving signals, such that the set of driving signals are provided to each switch of the ultrasound module;
the control and driving electronics being capable of controlling each switch, such that delivery of any one driving signal of the set of driving signals to a given ultrasound array element of the given ultrasound module is selectable, by actuation of the switch associated with the given ultrasound array element;
wherein, for each ultrasound module of the plurality of ultrasound modules:
   a number of driving signals in the set of driving signals provided to the ultrasound module is less than a number of ultrasound elements in the ultrasound module; and
   each driving signal of the set of driving signals has a respective phase, such that the set of driving signals has an associated set of phase values; and
wherein, for at least two ultrasound modules of the plurality of ultrasound modules, the set of phase values associated with the set of driving signals provided to the ultrasound module is a unique set of phase values customized to the ultrasound module;
wherein the control and driving electronics is configured to control each switch of each ultrasound module such that for each switch, the driving signal provided to the ultrasound element associated with the switch is the driving signal, from the set of driving signals provided to the switch, that has a phase value closest to an ideal phase associated with the ultrasound element for focusing ultrasound energy at a selected focus location.

In some example implementations of the system, the control and driving electronics are configured such that the unique set of phase values associated with a given ultrasound module is determined based on set of ideal phases respectively associated with the array elements of the given ultrasound module for focusing ultrasound energy at a plurality of locations within a selected region. The control and driving electronics may be configured such that the selected region is associated with a specific subject. The control and driving electronics are configured such that the selected region spans a planned target volume associated with the specific subject.

In some example implementations of the system, the control and driving electronics are configured such that the unique set of phase values associated with a given ultrasound module is determined based on a set of ideal phases respectively associated with the array elements of the given ultrasound module for focusing ultrasound energy at the selected focus location.

The control and driving electronics may be configured such that the unique set of phase values associated with a given ultrasound module include a maximum phase value, a minimum phase value, and at least one intermediate phase value lying between the maximum phase value and the minimum phase value, and wherein each intermediate phase value resides within a phase range spanning a maximum ideal phase and a minimum ideal phase of the set of ideal phases associated with the given ultrasound module.

The control and driving electronics may be configured such that the unique set of phase values associated with a given ultrasound module minimize an aggregate phase error measure, the aggregate phase error measure being determined based on phase errors associated with each ultrasound element of the given ultrasound module, each phase error being determined by calculating, for a given ultrasound element of the given ultrasound module, a difference between a phase value of a driving signal provided to the given ultrasound element and an ideal phase value associated with the given ultrasound element.

In some example implementations of the system, the control and driving electronics are configured such that each driving signal is a pulsed driving signal for generating pulsed ultrasound energy, and wherein each set of driving signals is delivered to a respective ultrasound module with a module-specific delay, each module-specific delay being selected to facilitate temporal alignment of the pulsed ultrasound energy from the ultrasound modules at the selected focus location.

In some example implementations of the system, the support is a conformal headset and wherein the selected focus location is an intracranial focus location.

In some example implementations of the system, for at least one ultrasound module, each driving signal is provided to an equal number of ultrasound elements.

In some example implementations of the system, for at least one ultrasound module, at least two driving signals are respectively provided to different numbers of ultrasound elements.

In some example implementations of the system, at least two ultrasound modules have different numbers of ultrasound elements.

In some example implementations of the system, at least two ultrasound modules are provided with different numbers of driving signals.

In some example implementations of the system, for at least two ultrasound modules, a number of driving signals provided to each ultrasound module is dependent on the number of ultrasound elements within the each ultrasound module and/or span of the ideal phase values.

In some example implementations of the system, for at least one module, a ratio of a number of ultrasound elements to a number of driving signals is at least 16.

In some example implementations of the system, the switches are optically configurable, and wherein the control and driving electronics comprises a light source controllable to deliver optical signals for configuring the switches to select suitable driving signals.

In some example implementations of the system, each unique set of phase values is determined based an acoustic model that characterizes spatial variations in acoustic properties of a tissue region that is to be insonified.

In another aspect, there is provided a method of generating focused ultrasound from an ultrasound system comprising a support and a plurality of ultrasound modules mechanically supported by the support, each ultrasound module comprising a respective array of ultrasound elements, wherein each ultrasound element is in electrical communication with an output of a respective switch uniquely associated with the ultrasound element, the method comprising:
for each ultrasound module:
   generating a set of driving signals for the ultrasound module, such that each driving signal has a respective phase value, wherein number of driving signals in the set of driving signals provided to the ultrasound module is less than a number of ultrasound elements in the ultrasound module;
   providing the set of driving signals to each switch of the ultrasound module; and
   controlling the switches of the ultrasound module such that for a given array element of the ultrasound module, the driving signal provided to the given array element is the driving signal from the set of driving signals that has a phase value that is closest to the ideal phase value associated with the array element focusing ultrasound energy at a selected focus location;
wherein, for at least two ultrasound modules of the plurality of ultrasound modules, the set of phase values associated with the set of driving signals provided to the ultrasound module is a unique set of phase values customized to the ultrasound module.

In some example implementations of the method, the unique set of phase values associated with a given ultrasound module is determined based on set of ideal phases respectively associated with the array elements of the given ultrasound module for focusing ultrasound energy at a plurality of locations within a selected region. The selected region may be associated with a specific subject. The selected region may span a planned target volume associated with the specific subject.

In some example implementations of the method, the unique set of phase values associated with a given ultrasound module is determined based on a set of ideal phases respectively associated with the array elements of the given ultrasound module for focusing ultrasound energy at the selected focus location.

The unique set of phase values associated with a given ultrasound module may include a maximum phase value, a minimum phase value, and at least one intermediate phase value lying between the maximum phase value and the minimum phase value, and wherein each intermediate phase value resides within a phase range spanning a maximum ideal phase and a minimum ideal phase of the set of ideal phases associated with the given ultrasound module.

The unique set of phase values associated with a given ultrasound module may minimize an aggregate phase error measure, the aggregate phase error measure being determined based on phase errors associated with each ultrasound element of the given ultrasound module, each phase error being determined by calculating, for a given ultrasound element of the given ultrasound module, a difference between a phase value of a driving signal provided to the given ultrasound element and an ideal phase value associated with the given ultrasound element.

In some example implementations of the method, each driving signal is a pulsed driving signal for generating pulsed ultrasound energy, and wherein each set of driving signals is delivered to a respective ultrasound module with a module-specific delay, each module-specific delay being selected to facilitate temporal alignment of the pulsed ultrasound energy from the ultrasound modules at the selected focus location.

In some example implementations of the method, the support is a conformal headset and wherein the selected focus location is an intracranial focus location.

In some example implementations of the method, each unique set of phases values is determined based an acoustic model that characterizes spatial variations in acoustic properties of a tissue region that is to be insonified.

In another aspect, there is provided a phased array ultrasound system comprising:
an array of ultrasound elements, wherein each ultrasound element is in electrical communication with an output of a respective switch uniquely associated with the ultrasound element; and
control and driving electronics configured to generate and deliver a set of driving signals and deliver the set of driving signals to each switch;
the control and driving electronics being capable of controlling each switch, such that delivery of any one driving signal of the set of driving signals to a given ultrasound array element is selectable, by actuation of the switch associated with the given ultrasound array element;
wherein a number of driving signals in the set of driving signals is less than a number of ultrasound elements in the array; and
wherein each driving signal of the set of driving signals has a respective phase, such that the set of driving signals has an associated set of phase values, the set of phases values being determined based on a set of ideal phases respectively associated with the array elements of the array for focusing ultrasound energy at one or more locations; and
wherein the control and driving electronics is configured to control each switch the driving signal provided to the ultrasound element associated with the switch is the driving signal, from the set of driving signals provided to the switch, that has a phase value closest to an ideal phase associated with the ultrasound element at a selected focus location.

In another aspect, there is provided a method of generating focused ultrasound from an array of ultrasound elements, wherein each ultrasound element is in electrical communication with an output of a respective switch uniquely associated with the ultrasound element, the method comprising:
generating a set of driving signals, each driving signal having a respective phase value, such that the set of driving signals has an associated set of phase values, wherein a number of driving signals in the set of driving signals is less than a number of ultrasound elements in the array of ultrasound elements, and wherein the set of phases values is determined based on a set of ideal phases respectively associated with the array elements of the array for focusing ultrasound energy at one or more locations;
providing the set of driving signals to each switch; and
controlling the switches such that for a given array element, the driving signal provided to the given array element is the driving signal from the set of driving signals that has a phase value that is closest to an ideal phase value associated with the array element for focusing ultrasound energy at a selected focus location.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1A shows an example ultrasound system with a reduced number of driving signals having pre-selected fixed phases.

FIG. 1B shows an example method of operating the system shown in FIG. 1A.

FIGS. 2A and 2B show the improved focusing obtainable using the system of FIG. 1A.

FIG. 3A shows an example improved ultrasound system with a reduced number of driving signals having phases determined based on a selected focal location.

FIG. 3B shows an example method of operating the system shown in FIG. 3A.

FIG. 4A shows an example modular ultrasound system employed a reduced number of driving signals having module-specific phases determined based on a selected focal location.

FIG. 4B shows an example method of selectively controlling switches of a given ultrasound module to deliver appropriate driving signals to each element.

FIG. 5 is a schematic of an example modular intracranial focused ultrasound system.

FIG. 6 is a photograph of another example modular intracranial focused ultrasound system, showing an inner view of a patient-customized ultrasound headset.

FIG. 7 shows an example system for performing transcranial diagnostic and/or therapeutic procedures.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

Unless defined otherwise, all technical and scientific terms used herein are intended to have the same meaning as commonly understood to one of ordinary skill in the art. Unless otherwise indicated, such as through context, as used herein, the following terms are intended to have the following meanings:

As noted above, when phased array devices are employed to delivery therapeutic ultrasound at frequencies in the range of 0.2 to 10 MHz, the need to maintain an inter-element spacing of less than A/2 translates into the requirement for an inter-element spacing of about 3 mm to 0.075 mm, with large therapeutic transducer arrays resulting in the need to deliver a large number of separate RF driving signals to the array elements, with an associated cost, size and complexity burden for the cable assembly and driving electronics. To overcome this problem, Caulfield et al. (R. E. Caulfield, X. Yin, J. Juste, and K. Hynynen, “A novel phase assignment protocol and driving system for a high-density focused ultrasound array,” IEEE Trans. Ultrason. Ferroelectr. Freq. Control, vol. 54, no. 4, pp. 793-801, 4/2007 2007) proposed that a therapeutic phased array ultrasound system could be simplified by reducing the number of RF driving signals.

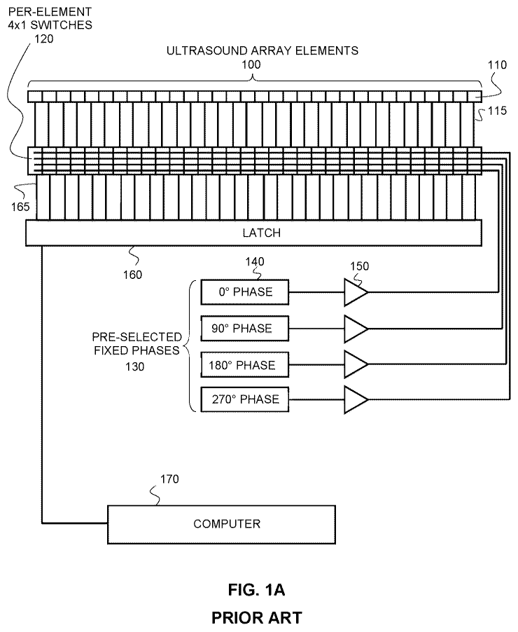

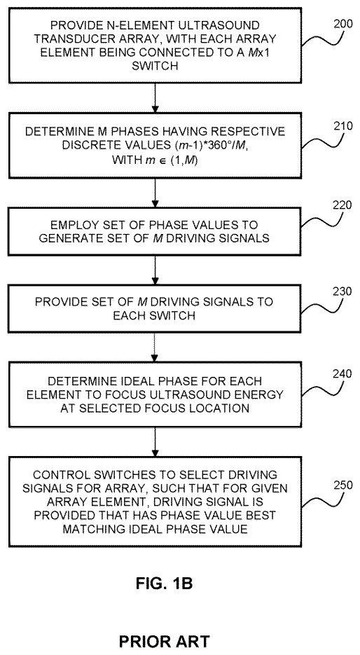

Caulfield proposed a configuration that is schematically illustrated in FIG. 1A and in the flow chart shown in FIG. 1B. As shown in FIG. 1A and step 200 of FIG. 1B, an N-element phased array 100 (N=32 in the illustrated example configuration) is provided such that each array element 110 is connected to the output of a respective Mx1 switch 120 (where M=4 in the illustrated example). As will be described below, each switch is employed to select a suitable driving signal from a reduced set of driving signals M, each driving signal having a different discrete phase, where M<N, such that a reduced set of driving signals are employed to generate focused ultrasound from the ultrasound array 100.

The reduced set of M phases for generating the driving signals are pre-determined, without consideration of a desired focal location or region, as shown at 130 in FIG. 1A and in step 210 of FIG. 1B, by dividing the unit circle into M phase increments, to obtain an evenly-spaced and pre-determined set of phases: (m−1)*360°/M, with m∈(1,M). In the example system shown in FIG. 1A, in which M=4, the discrete four phases are 0, 90°, 180°, and 270°.

As shown at step 220 of FIG. 1B, the M phases are then employed to generate M driving signals for driving the ultrasound array 100. FIG. 1A shows the generation of four driving signals 140, each with a different discrete (quantized) phase. The driving signals 140 are amplified via amplifiers 150.

The M driving signals are provided to each of the N switches 120, as shown in FIG. 1A and as per step 230 of FIG. 1B, such that each switch 120 is capable of delivering any one of the M driving signals to the array element connected to its output. In the example shown in FIG. 1, each of the 32 elements is connected, via a respective signal path 115, to a respective 4x1 switch that can be configured to deliver one of the four driving signals to the element.

The switches 120 are configured to select driving signals for delivery to the array elements 110 according to steps 240 and 250 in FIG. 1B. Firstly, an “ideal” phase is calculated for each array element 110 for generating a focus at a selected location if continuous phase selection was possible. For each element 110 of the array, the switch 120 associated with the element is programmed to select a suitable driving signal, from the M driving signals, by comparison of the “ideal” phase with the M phases associated with the M driving signals, selecting the driving signal having the closest phase to the ideal phase.

Caulfield's system implementation involved the use of a programmable latch 160 to provide the output lines for configuring the switches according to desired set of phases, with the latch being programmed by receiving phase selection data a computer 170. Although FIG. 1A shows a single connection line 165 between the latch and a given switch 120, Caulfield's system was based on the use of four switches per array element, with the outputs of the four switches connected in parallel to a given array element, with the four switches being configured by four connection lines provided by a latch 170. Furthermore, Caulfield employed multiple latches to configure the full set of array elements, with each latch being serially loaded.

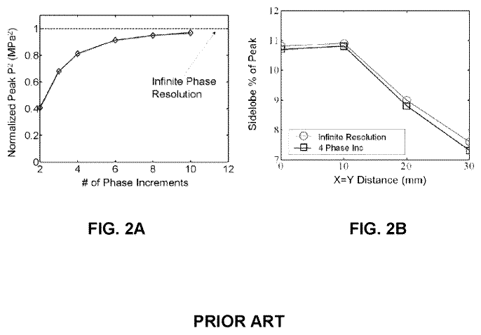

As can be seen from FIG. 1A, Caulfield's method employs a reduced set of M unique driving signals instead of requiring a continuous phase resolution and a full set of N driving signals to focus ultrasound energy at the selected focus. By connecting elements that have similar phase values to a single driving line, a significant reduction in the number of incoming lines can be achieved, thus greatly reducing the cost per channel while still providing the full benefits of the electronic beam steering and distortion compensation. Caufield showed that by connecting all of the transducer elements to four driving lines carrying 0, 90, 180 and 270 degree phase angles, a focal intensity could be achieved that exceeds 80% of what can be obtained with perfect phasing, as shown in FIGS. 2A and 2B.

However, the present inventor discovered that this performance could be further improved by employing an approach that is orthogonal to that of Caulfield. Indeed, while the approach of Caulfield involves the selection of a fixed, pre-determined set of phases for the M driving signals that is independent of the focus location within the tissue to be insonified, and also independent of spatial heterogeneities in the acoustic properties of the tissue (focus agnostic, and tissue acoustic heterogeneity agnostic), the present inventor realized that focusing performance of the ultrasound array could be further improved by employing (customizing) a set of phases M that is generated based on the selected focus location or based on focusing within a selected region, and optionally further based on spatial heterogeneities in the acoustic properties of the tissue, such that the phases could be computed to generate a reduced phase error and can therefore lead to an improved focal intensity. As will be described in further detail below, such an improvement can be particularly beneficial for practical 2D and 3D phased arrays that are modular in nature.

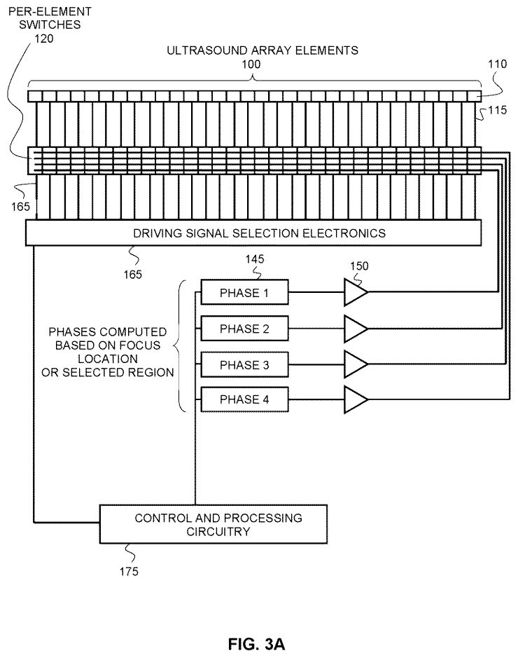

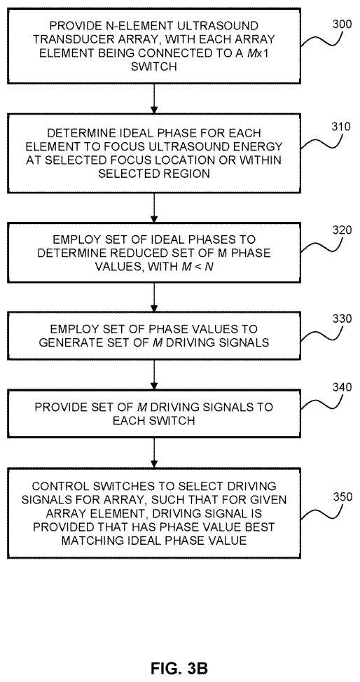

An example implementation of such an embodiment is illustrated in the example system shown in FIG. 3A and the flow chart shown in FIG. 3B. Referring first to FIG. 3A, and in stark contrast to the system of Caulfield shown in FIG. 1A, the four phases of the driving signals are computed based on the focus location or based on focusing within a selected region. As can be seen in the figure, the four phases are configured, for a given focal location, or for focusing with a selected region, via connection to control and processing circuitry 175 (examples of which are described in further detail below). Example methods for calculating the focal-location-dependent or focal region-dependent set of phases are described in further detail below.

The flowchart in FIG. 3B illustrates an example method of generating a set of M driving signals with M respective phases determined based on a selected focal location or selected region, and configuring the switches such that a suitable driving signal is delivered to each array element. Unlike the method of Caulfield, in which the selected focal location is only employed when calculating the ideal phase values that are employed to configure the switch for a given array element, to select a driving signal having a discrete pre-selected phase value that is closest to the ideal phase value, the method illustrated in FIG. 3B relies on the selected focus, or on a prescribed region in which focusing can occur, to determine the reduced set of phase M phase values, as shown in steps 310 and 320. The ideal phase values associated with the set of array elements, for focusing ultrasound energy from the array elements to a selected focus, or to a plurality of locations within a selected region, as determined in step 310, are employed to select a suitable set of M phase values in step 320. The number of phase values M (i.e. the number of driving signals) may be dependent on a span of the set of ideal phases.

In cases in which the ideal phase values are defined based on a selected region, as opposed to being defined based on a single desired focal location, the ideal phase values may be computed, for example, based on a plurality of focal locations that reside within a selected region (e.g. a set of representative focal locations that span, in a dense or sparse manner, the selected region). For example, a subset of ideal phase values may be computed for each location within the selected region, and the subsets of ideal phase values may be aggregated to define the final set of ideal phase values associated with the selected region.

The selected region may be defined in the absence of employing information associated with a particular subject or treatment plan, such as, for example, as a geometrical region defined relative to the ultrasound array. In other example embodiments, the selected region may be patient-specific and defined based on information associated with a particular subject or treatment plan. For example, the selected region may enclose, or lie within, a selected anatomical region, or a selected treatment target region, such as a tumor, or a region enclosing a tumor with a prescribed margin.

These M focal-location-specific or focal-region-specific phase values are then employed to generate the M respective driving signals, which are delivered to the switches in steps 330 and 340, with the switches being controlled in step 350 such that each array element is provided with a driving signal having a phase that best matches its respective ideal phase value.

The M phases may be generated from the set of ideal phases according to a wide range of implementations. The M phases may be generated based on the set of ideal phase values such that the focal intensity, at the selected focus, exceeds the focal intensity that would result from Caulfield's method of dividing the unit circle into M phase increments.

For example, the range of the ideal phase values (e.g. the maximum and minimum ideal phase values) may be employed to determine a phase range for generating the M phases. For example, in one implementation, the phase range corresponding to the ideal phase values may be divided into equal increments for generating the M phases. In some example embodiments, each of the M phases that lie between the maximum and minimum of the M phases are constrained to lie within the ideal phase range. In other example implementations, a nonlinear fitting method may be employed to select the M phase values. For example, some of the M phase values may be clustered in regions of the phase range that have a higher density of ideal phase values.

In some example implementations, an optimization algorithm may be employed to select the M phases based on the ideal phase values. For example, the M phase values may be selected to maximize the focal intensity when M driving signals respectively employing the M phases are delivered to the switches and the switches are configured to deliver, to respective array elements, driving signals having phase values that lie closest to the ideal phase values. In some example implementations, the M phase values may be selected as the phase values that minimize a measure, generated based on a phase error calculated as the difference between the ideal phase value and the closest of the M phase values, when summed over all array elements (such as a sum of squares of differences in phase values). It will be understood that many other methods may be employed to select the M phases based on the set of ideal phases.

Although the example system shown in FIG. 3A illustrates a specific case in which four phases (and respective driving signals) are calculated based on a selected focus location or plurality of focal locations within a selected region, it will be understood that the any number of phases (and respective driving signals) may be employed, provided that the number is less than the number of elements in the array.

Although a Mx1 switch is schematically illustrated as being connected to each array element 110, it will be understood that this functionality may be implemented according to a number of different configurations, such as, for example, a single Mx1 switch or as M 1x1 switches having outputs connected in parallel.

While FIG. 3A schematically illustrates an example configuration in which the switches 120 are programmed (configured) by intermediate driving signal selection electronics (e.g. a latch), it will be understood that the switches may be directly interfaced with a computing system such as control and processing circuitry 175.

Furthermore, although a single connection line is illustrated between the driving signal selection electronics (e.g. a programmable latch) and the switches 120, each switch may be configured according to multiple control lines connecting the switch to the driving signal electronics 160.

Because the setting of a given switch can be performed with only one or a few TTL lines (or wireless signals), the switching speed can be very high compared to more conventional methods of performing electronic focusing.

It will be understood that the switches may be any type of configurable switch, such as, for example, solid-state, mechanical or other types of switches such as an optically controlled switch. The switches can be made out of discrete components or can be application specific circuits (ASIC) specifically designed for the present application. Electronic control lines may be employed serially or in parallel to address each of the switches to set them to appropriate connection before each sonication. In some example embodiments, the switches may be wireless, such as, but not limited to, switches that can be activated by light, with light being delivered, for example, via optical fibers or other optical means such as laser beams or projections of light patterns.

The RF signals with the prescribed phase angles can be generated by a wide variety of means, including, but not limited to, oscillators, field programmable processors (FPC), and ASICs. The signals and their timing may be controlled by a central processing unit or via multiple subunits if a high switching speed is needed or beneficial.

In some example embodiments, the example embodiment illustrated in FIGS. 3A and 3B may be adapted for use with a modular phased array system. Such a modular phased array system includes a set of phased array ultrasound modules, each module including a respective array of ultrasound elements, with each ultrasound module being provided with a set of driving signals for generating and focused ultrasound according to the previously described methods. As described in further detail below, in some example implementations, at least two of the modules are provided with respective sets of driving signals that are module-specific, i.e. the discrete set of phases associated with each set of driving signals is customized or specific to a given module. At least two modules may have different numbers of array elements.

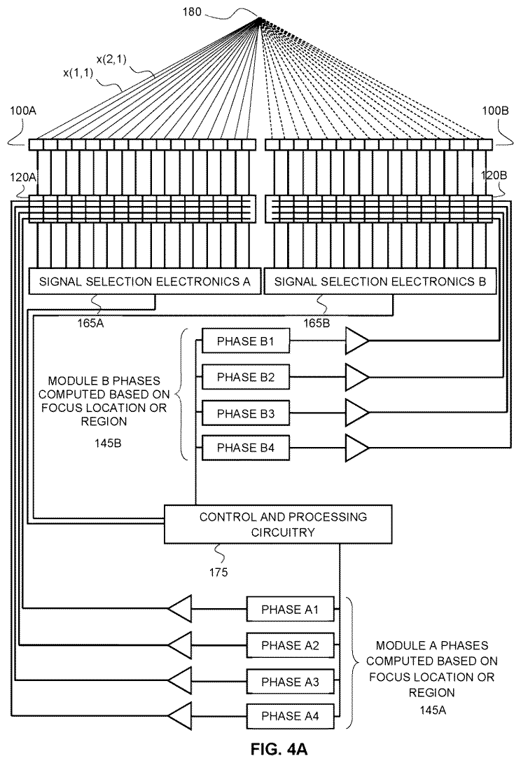

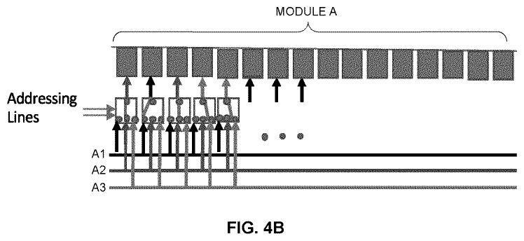

FIG. 4A schematically illustrates an example modular phased array ultrasound system that includes two modules: module A (100A) and module B (100B). Module A is provided with four different driving signals having respective phases A1, A2, A3 and A4, while module B is provided with four different driving signals having respective phases B1, B2, B3 and B4, such that the number of driving signals provided to each module is less than the number of array elements in each module.

As in the flow chart shown in FIG. 3B, the selected focus location, or a plurality of focal locations within a selected region (as described above), is employed to determine both the set of phases A1, A2, A3 and A4 for generating the driving signals sent to module A and the set of phases B1, B2, B3 and B4 for generating the driving signals sent to module B. The phases A1, A2, A3 and A4 are calculated based on a set of ideal phases associated with the elements of module A for focusing ultrasound energy at the selected focus location 180 or within a selected region. Likewise, the phases B1, B2, B3 and B4 are calculated based on a set of ideal phases associated with the elements of module B for focusing ultrasound energy at the selected focus location 180 or within a selected region.

Any of the example methods described above, or variations thereof, may be employed for computing the customized sets of phases for the modules. The phases employed for the driving signals provided to module A are thus customized to module A, and phases employed for the driving signals provided to module B are customized to module B, such that the set of phases A1, A2, A3 and A4 is different from the set of phases B1, B2, B3 and B4. While some example implementations may involve the calculation and use of customized sets of phases for each module, other example implementations may involve the calculation and use of customized sets of phases for any two or more of the modules within the array of modules.

As shown in the figure, each module has a respective set of switches 120A and 120B, with the switches being configured according to per-module signal selection electronics 165A and 165B, such that the driving signal delivered to a given array element of module A is the selected as the driving signal from the four driving signals (with phases A1, A2, A3 and A4) that has a phase value that is closest to an ideal phase value for the element, and such that the driving signal delivered to a given array element of module B is the selected as the driving signal from the four driving signals (with phases B1, B2, B3 and B4) that has a phase value that is closest to an ideal phase value for the element. The selection of suitable driving signals via the configuration of the various switches of module A is illustrated in FIG. 4B for an example implementation in which driving signals with only three different respective phases A1, A2 and A3 are provided to each switch of module A. It will be understood that for at least one ultrasound module, the driving signals may be selected such that at least two driving signals are respectively provided to different numbers of ultrasound array elements.

While FIG. 4A illustrates an example case in which each module has respective signal selection electronics 165A and 165B, it will be understood that these components can be merged into a single subsystem or integrated directly with the control and processing circuitry 175.

While FIG. 4A illustrates a non-liming example system with two modules, it will be understood that the present example embodiment may be adapted to a modular ultrasound system with any number of modules. More generally, in a modular phased array system with K modules, with each module being designated by k, with k∈[1,K], each module is provided with a respective set of Mₖ driving signals, with each driving signal having a respective phase φ_{m,k} with m∈[1,Mₖ]. Moreover, any two modules may have a different number of driving signals, for example, with Mₖ begin different for at least two different values of k. In some example implementations, the number of driving signals provided to each ultrasound module may be dependent on the number of ultrasound elements within each ultrasound module.

The ideal phases for each element of each transducer module can be determined by calculating the distance x(n, k) from the surface of each of the transducer elements in module k to the selected focus location 180 (or to a plurality of locations within a selected region), where n∈[1,N] counts the array elements within the module, as shown in FIG. 4A. If one assumes that the average ultrasound propagation speed in the medium is v and the ultrasound frequency is f, then the phase difference (φ) of each of the transducer elements, relative to for example element 1, can be obtained from equation:

$$ϕ ( n , k ) = 2 \prod ( x ( η , k ) - x ( 1 , k ) ) / ( v / f ) ,$$

where the phase is constrained to its principal value and is thus the wrapped phase.

After the ideal phases have been calculated for all of the transducer elements of a given module, the maximum and minimum ideal phases for the module can be employed to determine a range of phase that will be spanned by the M phase signals φ_{m,k}. Each of the elements of the module (n,k) are then connected, via their respective switches, to the driving signal with a phase φ_{m,k} that is closest to the ideal phase value for the element. A similar calculation can then be performed for the other ultrasound modules of the system.

If short sonications with the duration of only a few cycles are employed, then the sonication start time of each of the modules can be tailored such that the sound bursts will arrive to the indented target at the same time.

This time delay can be calculated from the average time of flight for each module. The time (t(n,k)) that the ultrasound burst travels from element n,k (of module k) to the selected focus location is: t(n,k)=x(n,k)/v. The average time of flight tₐᵥₑ for each module can be used to calculate the time delays needed for each module to assure that each of the sound burst arrive to the target at the same time. For example, if the tₐᵥₑ(2) of module 2 is the longest then the delays (d(k)) for the other modules can be calculated:

$$d ( k ) = {t}_{ave} ( k ) - {t}_{ave} ( 2 ) , where k = 1 to K ,$$

where K is the total number of modules. Accordingly, each of the modules can be individually controlled such that the start and end of the sonication from each module can be controlled to include relative delays in the sonication, thereby facilitating improved focusing.

While FIGS. 3A and 4A illustrates a one-dimensional transducer array, it will be understood that the present example embodiments may be adapted to two-dimensional transducer arrays.

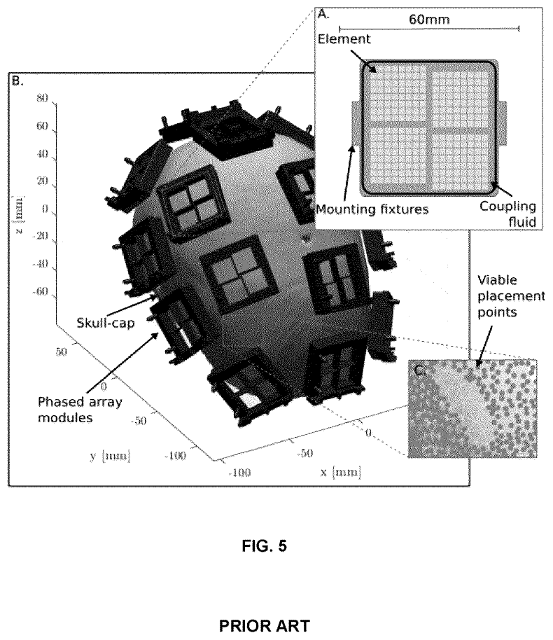

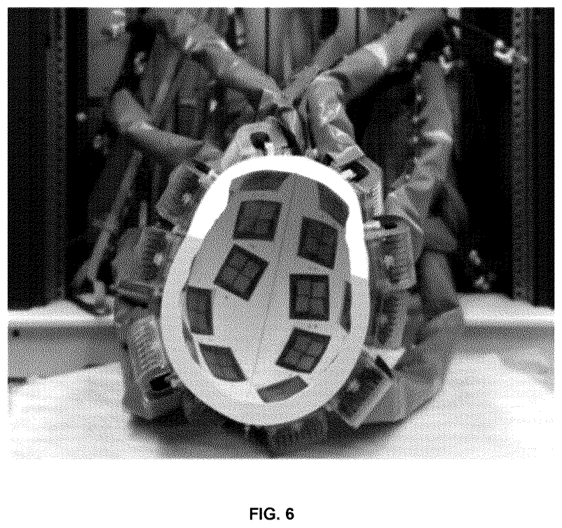

The example embodiments described above that pertain to modular ultrasound arrays can be particularly beneficial for applications involving the intracranial delivery of focused ultrasound. Examples of modular ultrasound devices for delivering focused intracranial ultrasound are illustrated in FIGS. 5 and 6. For example a conformal brain treatment system with 64 sub-arrays of 64 elements has been constructed and described by Adams (C. Adams et al., “Implementation of a Skull-Conformal Phased Array for Transcranial Focused Ultrasound Therapy,” (in eng), IEEE Trans Biomed Eng, vol. 68, no. 11, pp. 3457-3468 November 2021, doi: 10.1109/tbme.2021.3077802).

Such systems could be adapted to employ the embodiments of the present disclosure to reduce the number of driving signals that are delivered to the ultrasound modules, while still facilitating accurate focusing. In such cases, by selectively optimizing the set of phases employed to generate a reduced number of driving signals for each module (or at least two modules), the system complexity can be significantly reduced.

It is noted that an optimization algorithm employed to select the different sets of phases for each module can advantageously take into account tissue heterogeneities that may distort the ultrasound propagation such as skull bone for trans-skull sonications. For example, the ideal phases can be determined based on an acoustic model that characterizes spatial variations in acoustic properties of the tissue region that is to be insonified. For example, image data (e.g. MR, CT, optical, x-ray, and ultrasound) can be processed to identify subregions with known tissue types within the region that is to be insonified, and a model can be generated by associating, with each subregion, known acoustic properties corresponding to the tissue type of the subregion. In one example implementation, the ideal phase calculation can take into account tissue heterogeneity, such as skull bone, using data derived from CT scans by first segmenting the CT scan to identify known tissue types and then constructing an acoustic model that uses speed of sound values for the various tissue types from literature and density values from the calibrated CT scans. For the skull bone the speed of sound and attenuation are density depended as was shown by Clement and Hynynen (G. T. Clement and K. Hynynen, “A non-invasive method for focusing ultrasound through the human skull,” Phys. Med Biol, vol. 47, no. 8, pp. 1219-1236, Apr. 21, 2002 2002; G. T. Clement and K. Hynynen, “Correlation of ultrasound phase with physical skull properties,” Ultrasound Med Biol, vol. 28, no. 5, pp. 617-624, 5/2002 2002) and Pichardo et al. (S. Pichardo, V. W. Sin, and K. Hynynen, “Multi-frequency characterization of the speed of sound and attenuation coefficient for longitudinal transmission of freshly excised human skulls,” Phys Med Biol, vol. 56, no. 1, pp. 219-250, Jan. 7, 2011 2011). The propagation of shear waves can also be modeled (e.g. G. T. Clement, P. J. White, and K. Hynynen, “Enhanced ultrasound transmission through the human skull using shear mode conversion,” J. Acoust. Soc. Am, vol. 115, no. 3, pp. 1356-1364, 3/2004 2004. And P. J. White, G. T. Clement, and K. Hynynen, “Longitudinal and shear mode ultrasound propagation in human skull bone,” Ultrasound Med. Biol, vol. 32, no. 7, pp. 1085-1096, 7/2006 2006). The wave propagation can be modeled either using ray tracing method, spectral methods, full-wave simulation or inverse methods using a point source in the indented focus and then propagating the wave to each of the array elements. For each of these methods the relative phase shift or delay can be calculated relative to a reference signal. After the phases for each of the elements have been determined the phases of the driving signals will be determined to minimize the combined phase error between element phases and the driving signal phases.

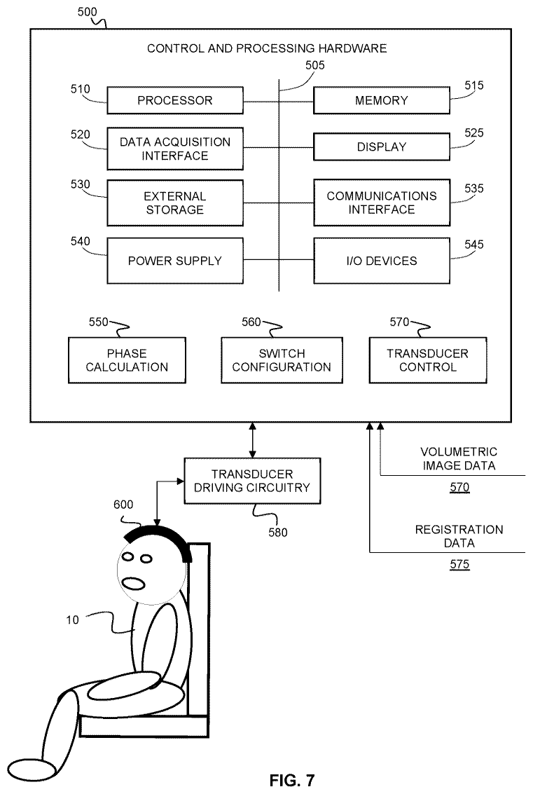

FIG. 7 provides a block diagram illustrating an example implementation of a system for performing diagnostic or therapeutic transcranial procedures. Control and processing hardware 500 is operably connected to a transcranial headset 600, optionally via transducer driver electronics/circuitry 580.

The control and processing hardware 500, which includes one or more processors 510 (for example, a CPU/microprocessor), bus 505, memory 515, which may include random access memory (RAM) and/or read only memory (ROM), a data acquisition interface 520, a display 525, external storage 530, one more communications interfaces 535, a power supply 540, and one or more input/output devices and/or interfaces 545 (e.g. a speaker, a user input device, such as a keyboard, a keypad, a mouse, a position tracked stylus, a position tracked probe, a foot switch, and/or a microphone for capturing speech commands).

Volumetric image data 570 and transducer registration data 575 may be stored on an external database or stored in memory 515 or storage 530 of control and processing hardware 500.

The control and processing hardware 500 may be programmed with programs, subroutines, applications or modules, which include executable instructions, which when executed by the one or more processors 510, causes the system to perform one or more methods described in the present disclosure. Such instructions may be stored, for example, in memory 515 and/or other storage.

In the example embodiment shown, module 550 is employed to calculate the set of phases for generating the reduced set of driving signals for each ultrasound module, and module 560 is employed to control the switches of each ultrasound module to select an appropriate driving signal from for each array element. The transducer control module 560 includes executable instructions for controlling the transducers of the transcranial headset 600 to deliver energy to a target location or region of interest, based on the registration of the transducer positions and orientations with the volumetric image data as per the transducer registration data 575, and based on the delivery of driving signals provided to each module as per the methods described above. For example, the transcranial headset 600 may support a plurality of phased-array transducers, and transducer control module 555 may control the beamforming applied (on transmit and/or receive) to deliver, based on the known positions and orientations of the phased array transducers relative to the volumetric image data, one or more focused energy beams to a region of interest. The region of interest may be specified intraoperatively by a user (e.g. via a user interface controlled by control and processing hardware 500) or according to a pre-established surgical plan.

Although only one of each component is illustrated in FIG. 7, any number of each component can be included in the control and processing hardware 500. For example, a computer typically contains a number of different data storage media. Furthermore, although bus 505 is depicted as a single connection between all of the components, it will be appreciated that the bus 505 may represent one or more circuits, devices or communication channels which link two or more of the components. For example, in personal computers, bus 505 often includes or is a motherboard. Control and processing hardware 500 may include many more or less components than those shown.

The control and processing hardware 500 may be implemented as one or more physical devices that are coupled to processor 510 through one of more communications channels or interfaces. For example, control and processing hardware 500 can be implemented using application specific integrated circuits (ASICs). Alternatively, control and processing hardware 500 can be implemented as a combination of hardware and software, where the software is loaded into the processor from the memory or over a network connection.

Some aspects of the present disclosure can be embodied, at least in part, in software, which, when executed on a computing system, transforms a computing system into a specialty-purpose computing system that is capable of performing the methods disclosed herein. That is, the techniques can be carried out in a computer system or other data processing system in response to its processor, such as a microprocessor, executing sequences of instructions contained in a memory, such as ROM, volatile RAM, non-volatile memory, cache, magnetic and optical disks, or a remote storage device. Further, the instructions can be downloaded into a computing device over a data network in a form of compiled and linked version. Alternatively, the logic to perform the processes as discussed above could be implemented in additional computer and/or machine readable media, such as discrete hardware components as large-scale integrated circuits (LSI's), application-specific integrated circuits (ASIC's), or firmware such as electrically erasable programmable read-only memory (EEPROM's) and field-programmable gate arrays (FPGAs).

A computer readable medium can be used to store software and data which when executed by a data processing system causes the system to perform various methods. The executable software and data can be stored in various places including for example ROM, volatile RAM, non-volatile memory and/or cache. Portions of this software and/or data can be stored in any one of these storage devices. In general, a machine readable medium includes any mechanism that provides (i.e., stores and/or transmits) information in a form accessible by a machine (e.g., a computer, network device, personal digital assistant, manufacturing tool, any device with a set of one or more processors, etc.).

Examples of computer-readable media include but are not limited to recordable and non-recordable type media such as volatile and non-volatile memory devices, read only memory (ROM), random access memory (RAM), flash memory devices, floppy and other removable disks, magnetic disk storage media, optical storage media (e.g., compact discs (CDs), digital versatile disks (DVDs), etc.), among others. The instructions can be embodied in digital and analog communication links for electrical, optical, acoustical or other forms of propagated signals, such as carrier waves, infrared signals, digital signals, and the like. As used herein, the phrases “computer readable material” and “computer readable storage medium” refer to all computer-readable media, except for a transitory propagating signal per se.

It will be understood that any of the present example embodiments may be adapted for the generation of ultrasound focused ultrasound for a wide range of clinical and research applications that are not limited to intracranial therapy.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1. A phased array ultrasound system comprising:
a support;
a plurality of ultrasound modules mechanically supported by said support, each ultrasound module comprising a respective array of ultrasound elements, wherein each ultrasound element is in electrical communication with an output of a respective switch uniquely associated with said ultrasound element; and
control and driving electronics configured to generate and deliver, to each ultrasound module, a respective set of driving signals, such that the set of driving signals are provided to each switch of said ultrasound module;
said control and driving electronics being capable of controlling each switch, such that delivery of any one driving signal of the set of driving signals to a given ultrasound array element of said given ultrasound module is selectable, by actuation of the switch associated with said given ultrasound array element;
wherein, for each ultrasound module of the plurality of ultrasound modules:
a number of driving signals in the set of driving signals provided to said ultrasound module is less than a number of ultrasound elements in said ultrasound module; and
each driving signal of the set of driving signals has a respective phase, such that the set of driving signals has an associated set of phase values; and
wherein, for at least two ultrasound modules of the plurality of ultrasound modules, the set of phase values associated with the set of driving signals provided to the ultrasound module is a unique set of phase values customized to the ultrasound module;
wherein said control and driving electronics is configured to control each switch of each ultrasound module such that for each switch, the driving signal provided to the ultrasound element associated with said switch is the driving signal, from the set of driving signals provided to said switch, that has a phase value closest to an ideal phase associated with said ultrasound element for focusing ultrasound energy at a selected focus location.

2. The phased array ultrasound system according to claim 1 wherein said control and driving electronics are configured such that the unique set of phase values associated with a given ultrasound module is determined based on set of ideal phases respectively associated with said array elements of said given ultrasound module for focusing ultrasound energy at a plurality of locations within a selected region.

3. The phased array ultrasound system according to claim 2 wherein said control and driving electronics are configured such that the selected region is associated with a specific subject.

4. The phased array ultrasound system according to claim 3 wherein said control and driving electronics are configured such that the selected region spans a target volume associated with the specific subject.

5. The phased array ultrasound system according to any one of claims 1 to 4 wherein, for at least two ultrasound modules, a number of driving signals provided to each ultrasound module is dependent on a span of the set of ideal phases.

6. The phased array ultrasound system according to claim 1 wherein said control and driving electronics are configured such that the unique set of phase values associated with a given ultrasound module is determined based on a set of ideal phases respectively associated with said array elements of said given ultrasound module for focusing ultrasound energy at the selected focus location.

7. The phased array ultrasound system according to claim 6 wherein said control and driving electronics are configured such that the unique set of phase values associated with a given ultrasound module include a maximum phase value, a minimum phase value, and at least one intermediate phase value lying between the maximum phase value and the minimum phase value, and wherein each intermediate phase value resides within a phase range spanning a maximum ideal phase and a minimum ideal phase of the set of ideal phases associated with the given ultrasound module.

8. The phased array ultrasound system according to claim 6 wherein said control and driving electronics are configured such that the unique set of phase values associated with a given ultrasound module minimize an aggregate phase error measure, the aggregate phase error measure being determined based on phase errors associated with each ultrasound element of said given ultrasound module, each phase error being determined by calculating, for a given ultrasound element of the given ultrasound module, a difference between a phase value of a driving signal provided to said given ultrasound element and an ideal phase value associated with said given ultrasound element.

9. The phased array ultrasound system according to any one of claims 6 to 8 wherein, for at least two ultrasound modules, a number of driving signals provided to each ultrasound module is dependent on a span of the set of ideal phases.

10. The phased array ultrasound system according to any one of claims 1 to 9 wherein said control and driving electronics are configured such that each driving signal is a pulsed driving signal for generating pulsed ultrasound energy, and wherein each set of driving signals is delivered to a respective ultrasound module with a module-specific delay, each module-specific delay being selected to facilitate temporal alignment of the pulsed ultrasound energy from said ultrasound modules at the selected focus location.

11. The phased array ultrasound system according to any one of claims 1 to 10 wherein said support is a conformal headset and wherein the selected focus location is an intracranial focus location.

12. The phased array ultrasound system according to any one of claims 1 to 11 wherein, for at least one ultrasound module, each driving signal is provided to an equal number of ultrasound elements.

13. The phased array ultrasound system according to any one of claims 1 to 11 wherein, for at least one ultrasound module, at least two driving signals are respectively provided to different numbers of ultrasound elements.

14. The phased array ultrasound system according to any one of claims 1 to 11 wherein at least two ultrasound modules have different numbers of ultrasound elements.

15. The phased array ultrasound system according to any one of claims 1 to 11 wherein at least two ultrasound modules are provided with different numbers of driving signals.

16. The phased array ultrasound system according to any one of claims 1 to 11 wherein, for at least two ultrasound modules, a number of driving signals provided to each ultrasound module is dependent on the number of ultrasound elements within said each ultrasound module.

17. The phased array ultrasound system according to any one of claims 1 to 16 wherein, for at least one module, a ratio of a number of ultrasound elements to a number of driving signals is at least 16.

18. The phased array ultrasound system according to any one of claims 1 to 17 wherein said switches are optically configurable, and wherein said control and driving electronics comprises a light source controllable to deliver optical signals for configuring said switches to select suitable driving signals.

19. The phased array ultrasound system according to any one of claims 1 to 18 wherein each unique set of phase values is determined based an acoustic model that characterizes spatial variations in acoustic properties of a tissue region that is to be insonified.

20. A method of generating focused ultrasound from an ultrasound system comprising a support and a plurality of ultrasound modules mechanically supported by the support, each ultrasound module comprising a respective array of ultrasound elements, wherein each ultrasound element is in electrical communication with an output of a respective switch uniquely associated with the ultrasound element, the method comprising:
for each ultrasound module:
generating a set of driving signals for the ultrasound module, such that each driving signal has a respective phase value, wherein number of driving signals in the set of driving signals provided to the ultrasound module is less than a number of ultrasound elements in the ultrasound module;
providing the set of driving signals to each switch of the ultrasound module; and
controlling the switches of the ultrasound module such that for a given array element of the ultrasound module, the driving signal provided to the given array element is the driving signal from the set of driving signals that has a phase value that is closest to the ideal phase value associated with the array element focusing ultrasound energy at a selected focus location;
wherein, for at least two ultrasound modules of the plurality of ultrasound modules, the set of phase values associated with the set of driving signals provided to the ultrasound module is a unique set of phase values customized to the ultrasound module.

21. The method according to claim 20 wherein the unique set of phase values associated with a given ultrasound module is determined based on set of ideal phases respectively associated with said array elements of said given ultrasound module for focusing ultrasound energy at a plurality of locations within a selected region.

22. The method according to claim 21 wherein the selected region is associated with a specific subject.

23. The method according to claim 22 wherein the selected region spans a target volume associated with the specific subject.

24. The method according to claim 20 wherein the unique set of phase values associated with a given ultrasound module is determined based on a set of ideal phases respectively associated with the array elements of the given ultrasound module for focusing ultrasound energy at the selected focus location.

25. The method according to claim 21 wherein the unique set of phase values associated with a given ultrasound module include a maximum phase value, a minimum phase value, and at least one intermediate phase value lying between the maximum phase value and the minimum phase value, and wherein each intermediate phase value resides within a phase range spanning a maximum ideal phase and a minimum ideal phase of the set of ideal phases associated with the given ultrasound module.

26. The method according to claim 21 wherein the unique set of phase values associated with a given ultrasound module minimize an aggregate phase error measure, the aggregate phase error measure being determined based on phase errors associated with each ultrasound element of the given ultrasound module, each phase error being determined by calculating, for a given ultrasound element of the given ultrasound module, a difference between a phase value of a driving signal provided to said given ultrasound element and an ideal phase value associated with said given ultrasound element.

27. The method according to any one of claims 20 to 26 wherein each driving signal is a pulsed driving signal for generating pulsed ultrasound energy, and wherein each set of driving signals is delivered to a respective ultrasound module with a module-specific delay, each module-specific delay being selected to facilitate temporal alignment of the pulsed ultrasound energy from the ultrasound modules at the selected focus location.

28. The method according to any one of claims 20 to 27 wherein the support is a conformal headset and wherein the selected focus location is an intracranial focus location.

29. The method according to any one of claims 20 to 28 wherein each unique set of phases values is determined based an acoustic model that characterizes spatial variations in acoustic properties of a tissue region that is to be insonified.

30. A phased array ultrasound system comprising:
an array of ultrasound elements, wherein each ultrasound element is in electrical communication with an output of a respective switch uniquely associated with said ultrasound element; and
control and driving electronics configured to generate and deliver a set of driving signals and deliver the set of driving signals to each switch;
said control and driving electronics being capable of controlling each switch, such that delivery of any one driving signal of the set of driving signals to a given ultrasound array element is selectable, by actuation of the switch associated with said given ultrasound array element;
wherein a number of driving signals in the set of driving signals is less than a number of ultrasound elements in said array; and
wherein each driving signal of the set of driving signals has a respective phase, such that the set of driving signals has an associated set of phase values, the set of phases values being determined based on a set of ideal phases respectively associated with said array elements of said array for focusing ultrasound energy at one or more locations; and
wherein said control and driving electronics is configured to control each switch the driving signal provided to the ultrasound element associated with said switch is the driving signal, from the set of driving signals provided to said switch, that has a phase value closest to an ideal phase associated with said ultrasound element at a selected focus location.

31. A method of generating focused ultrasound from an array of ultrasound elements, wherein each ultrasound element is in electrical communication with an output of a respective switch uniquely associated with the ultrasound element, the method comprising:
generating a set of driving signals, each driving signal having a respective phase value, such that the set of driving signals has an associated set of phase values, wherein a number of driving signals in the set of driving signals is less than a number of ultrasound elements in the array of ultrasound elements, and wherein the set of phases values is determined based on a set of ideal phases respectively associated with the array elements of said array for focusing ultrasound energy at one or more locations;
providing the set of driving signals to each switch; and
controlling the switches such that for a given array element, the driving signal provided to the given array element is the driving signal from the set of driving signals that has a phase value that is closest to an ideal phase value associated with the array element for focusing ultrasound energy at a selected focus location.
